(19) United States
(12) Patent Application Publication
(10) Pub. No.: US 2004/0197293 A1
(43) Pub. Date: Oct. 7, 2004

(54) Anionic polyurethanes with elastic property

(76) Inventor: Nathalie Mougin, Paris (FR)

Correspondence Address: Finnegan Henderson Farabow, Garrett & Dunner, 1300 I Street NW, Washington, DC, 20005, US

(21) Appl. No.: 10/469,785

(22) PCT Filed: Feb. 27, 2002
(86) PCT No.: PCT/FR02/00703

(30) Foreign Application Priority Data
Mar. 5, 2001 (FR) 01/02947

(51) Int. Cl. A61K 7/06; A61K 7/11; C08G 18/32; C08G 18/60; C08G 18/28

(52) U.S. Cl. 424/70.17; 528/44; 528/68; 528/72

(57) ABSTRACT

The invention concerns novel anionic polyurethanes with elastic property, that is having an instantaneous elastic recovery ranging between 5% and 100% consisting essentially (a1) of anionic units derived from at least a monomer or polymer compound with sulphonic and/or phosphonic acid function and having at least two functions reactive to labile hydrogen, optionally (a2) non-ionic units derived from at least a non-ionic monomer or polymer compound having at least two functions reactive to labile hydrogen, and (b) units derived from at least a diisocyanate, provided that at least one type of the units (a1) and (a2) is derived from a polymer having a glass transition temperature (Tg) measured by differential enthalpy analysis, less than 10° C. and that said sequences with Tg less than 10° C. represent 20 to 90% of the total weight of the polyurethane. The invention also concerns cosmetic compositions containing the novel anionic polyurethanes with elastic property.

The present invention relates to novel anionic polyurethanes with elastic property and to their use in cosmetic compositions.

The formation of deposits and of films with elastic properties has always been the subject of major research studies in cosmetics. Indeed, most of the areas of the human body capable of receiving cosmetic deposits, such as the skin, the lips, the hair, the eyelashes and the nails, are subjected to high mechanical strains and stresses. Cosmetic films and deposits should be able to withstand these stresses and follow these strains without breaking.

The use of polyurethanes in cosmetics has been known for a long time and is described for example in patents and patent applications WO 94/13724, WO 94/03510, WO 96/14049, EP 0 214 626, U.S. Pat. No. 5,626,840, DE 42 25 045, U.S. Pat. No. 4,743,673 and EP 0 619 111.

The polyurethanes disclosed in these documents have nevertheless glass transition temperatures (T_{g}) greater than or close to room temperature, that is to say that at room temperature they are in the glassy state and form brittle films which are unacceptable for a cosmetic application.

There are of course physiologically acceptable polymers having low glass transition temperatures, such as for example acrylic polymers, but these polymers generally form very sticky deposits, which is a disadvantage in most cosmetic applications.

The applicant has discovered a novel group of physiologically acceptable polyurethanes which form films which are nonsticky, nonbrittle and capable of plastic and elastic deformations. These advantageous viscoelastic properties are due to the presence, in the polymer, of long macromolecular units having a relatively low glass transition temperature and which, as a result, are not in the glassy state at room temperature.

The subject of the present invention is consequently anionic polyurethanes with elastic property, essentially consisting

(a1) of anionic units derived from at least one monomer or polymer compound with sulfonic and/or phosphonic acid function and having at least two reactive functions with labile hydrogen, optionally

(a2) nonionic units derived from at least one nonionic monomer or polymer compound having at least two reactive functions with labile hydrogen, and

(b) units derived from at least one diisocyanate,

it being understood that at least one type of units (a1) and (a2) is derived from a polymer having a glass transition temperature (Tg), measured by differential scanning calorimetry, less than 10° C. and that these sequences with Tg less than 10° C. represent from 20% to 90% of the total weight of the polyurethane.

The subject of the invention is also the use of the above anionic polyurethanes with elastic property in cosmetic compositions in order to improve the viscoelastic properties of the cosmetic deposits and films obtained from these compositions.

Its subject is in particular the use of these polyurethanes in hair styling lacquers and compositions, in nail varnishes and in makeup compositions.

The subject of the invention is also cosmetic compositions containing the above anionic polyurethanes with elastic properties.

The use of the anionic polyurethanes with elastic properties of the present invention in hair styling lacquers and compositions makes it possible to improve the suppleness of the hairstyle, that is to say to obtain an elastic behavior for the hair which is more natural than that obtained with the usual fixing polymers.

The anionic nature of the novel polyurethanes of the present invention makes them moreover very easy to remove by simply washing the hair.

It is possible to use these polyurethanes to cover the nails with a glossy protective film which is resistant to mechanical attacks. Their incorporation into nail varnishes improves the impact resistance thereof and delays flaking.

The above anionic polyurethanes may also be used to improve the staying power of makeup compositions for the skin, the lips and the superficial body growths. The deposits obtained follow the deformations of the keratinous substrates and do not pull the skin.

In all these applications, nonsticky products are obtained.

The presence of the anionic fillers confers on the polyurethanes of the present invention a fairly hydrophilic nature independent of the pH of the medium containing them. The anionic polyurethanes of the present invention are consequently soluble, or at least dispersible, in polar solvents and in particular in water and lower alcohols, which greatly facilitates their formulation in cosmetic compositions.

As indicated above, the anionic polyurethanes with elastic property of the present invention essentially consist of three types of unit which are

(a1) anionic units derived from at least one monomer or polymer compound with sulfonic and/or phosphonic acid function and having at least two reactive functions with labile hydrogen,

(a2) optional nonionic units derived from at least one nonionic monomer or polymer compound having at least two reactive functions with labile hydrogen, and

(b) units derived from at least one diisocyanate.

As used in the present invention, the terms “sulfonic acid function” and “phosphonic acid function” denote not only the protonated acid form of these functions but also the forms partially or completely neutralized with a base, that is to say the sulfonate (—SO₃⁻), phosphonate (—PO₃H⁻) and diphosphonate (PO₃⁻²) groups.

The expression reactive functions with labile hydrogen is understood to mean functions which are capable, after departure of a hydrogen atom, of forming covalent bonds with the isocyanate functions of the compounds forming the units (b). There may be mentioned by way of example of such functions hydroxyl, primary amine (—NH₂) or secondary amine (—NHR) groups, or alternatively thiol (—SH) groups.

The polycondensation of compounds carrying these reactive functions with labile hydrogen with diisocyanates gives, according to the nature of the reactive functions carrying the labile hydrogen (—OH, —NH₂, —NHR or —SH), polyurethanes in the strict sense, polyureas or polythiourethanes, respectively. All these polymers are grouped together in the present application, for the sake of simplicity, under the term polyurethanes.

When the compounds with sulfonic and/or phosphonic acid function forming the units (a1) carry more than two functions with labile hydrogen, the polyurethanes obtained have a branched, optionally even crosslinked, structure.

In a preferred embodiment of the polyurethanes of the present invention, the compounds with sulfonic and/or phosphonic acid function forming the anionic units (a1) have only two reactive functions with labile hydrogen and the polyurethanes obtained by polycondensation consequently have an essentially linear structure.

It is of course also possible to use a mixture of difunctional compounds containing a low proportion of compounds with sulfonic and/or phosphonic acid function carrying more than two reactive functions with labile hydrogen.

The compounds with sulfonic and/or phosphonic acid function forming the anionic units (a1) are preferably chosen from the compounds corresponding to one of the following formulae:

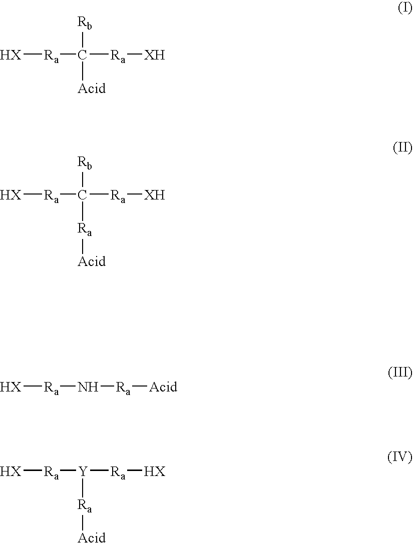

in which

Acid represents a sulfonic acid or a phosphonic acid group, in protonated or salified form,

each Rₐ independently represents a direct bond or a linear or branched C₁₋₆ alkylene group, a C₃₋₆ cyclo-alkylene group or an arylene group, it being possible for all to be substituted with one or more halogen atoms and to contain one or more heteroatoms chosen from O, P and S,

R_{b} represents a hydrogen atom or an alkyl group which may contain one or more heteroatoms chosen from O, P and S,

Y represents a saturated, unsaturated or aromatic, cyclic C₅₋₁₀ group optionally containing one or more heteroatoms chosen from O, P and S,

each X independently represents an oxygen or a sulfur atom or an NH or NR_{c} group, where R_{c} represents a C₁₋₆ alkyl group.

In a preferred embodiment of the anionic polyurethanes with elastic property of the present invention, the reactive functions with labile hydrogen are amine functions, that is to say in the formulae (I) to (IV) above X═NH or NR_{c}, giving polymers of the polyurea type. The polyureas indeed form films which are distinguishable by an excellent cohesion which improves the retention of the hairstyle or the abrasion resistance of the nail varnishes containing these polyureas.

There may also be mentioned, by way of example of compounds with sulfonic and/or phosphonic acid function forming the anionic units (a1)

1,1-diaminomethanesulfonic acid,

diaminobenzenesulfonic acids,

1,1-di(hydroxymethyl)ethanesulfonic acid,

1,1-di(hydroxyethyl)ethanesulfonic acid,

1,1-di(hydroxymethyl)propanesulfonic acid,

1,1-di(hydroxymethyl)methanesulfonic acid,

1,1-di(hydroxypropyl)ethanesulfonic acid,

1,1-di(hydroxyoctyl)ethanesulfonic acid,

3-(2,3-dihydroxypropoxy)propanesulfonic acid,

3-[2,2-bis(hydroxymethyl)butoxy]-2-methyl-1-propanesulfonic acid,

1,1-di(aminoethyl)ethanesulfonic acid,

1,1-diaminomethanesulfonic acid,

1,1-diaminopropanesulfonic acid,

0,1,2-diaminopropanesulfonic acid,

1,1-di(mercaptoethyl)ethanesulfonic acid,

1,1-dimercaptomethanesulfonic acid,

1,1-dimercaptopropanesulfonic acid,

1,2-dimercaptohexanesulfonic acid,

1-amino-1-hydroxymethylethanesulfonic acid,

1-amino-1-hydroxyethylethanesulfonic acid,

1-amino-1-hydroxymethylpropanesulfonic acid,

1-amino-1-hydroxymethylmethanesulfonic acid,

1-amino-1-hydroxypropylethanesulfonic acid,

2-amino-1-hydroxypropylethanesulfonic acid,

1-amino-1-hydroxyoctylethanesulfonic acid,

1-hydroxymethyl-1-mercaptoethanesulfonic acid,

1-hydroxyethyl-2-mercaptoethylethanesulfonic acid,

1-hydroxymethyl-1-mercaptopropanesulfonic acid,

1-hydroxypropyl-1-mercaptomethanesulfonic acid,

1-hydroxypropyl-1-mercaptoethanesulfonic acid,

2-hydroxyethyl-1-mercaptomethylethanesulfonic acid,

1-hydroxybutyl-1-mercaptooctylethanesulfonic acid,

1-amino-1-mercaptomethylethanesulfonic acid,

1-amino-1-mercaptoethylethanesulfonic acid,

1-amino-1-mercaptomethylpropanesulfonic acid,

1-amino-1-mercaptomethylmethanesulfonic acid,

1-amino-1-mercaptopropylethanesulfonic acid,

2-amino-1-mercaptopropylethanesulfonic acid,

1-amino-1-mercaptooctylethanesulfonic acid,

1-amino-1-mercaptopropylethylphosphonic acid,

dimercaptomethylphosphonic acid,

dimercaptoethylphosphonic acid,

dimercaptopropylphosphonic acid,

1-amino-1-hydroxymethylethylphosphonic acid,

1-amino-1-(hydroxyethyl)ethylphosphonic acid,

1-amino-1-(hydroxymethyl)propylphosphonic acid,

1-amino-1-(hydroxymethyl)methylphosphonic acid,

1-amino-1-(hydroxypropyl)ethylphosphonic acid,

2-amino-1-(hydroxypropyl)ethylphosphonic acid,

1-amino-1-(hydroxyoctyl)ethylphosphonic acid,

1-hydroxymethyl-1-mercaptothylphosphonic acid,

1-hydroxyethyl-2-(mercaptoethyl)ethylphosphonic acid,

1-hydroxymethyl-1-mercaptopropylphosphonic acid,

1-hydroxypropyl-1-mercaptomethylphosphonic acid,

1-hydroxypropyl-1-(mercaptobutyl)ethylphosphonic acid,

2-hydroxyethyl-1-(mercaptomethyl)ethylphosphonic acid,

1-hydroxybutyl-1-(mercaptooctyl)ethylphosphonic acid,

1-amino-1-(mercaptomethyl)ethylphosphonic acid,

1-amino-1-(mercaptoethyl)butylphosphonic acid,

1-amino-1-mercaptopropylphosphonic acid,

1-amino-1-(mercaptomethyl)ethylphosphonic acid,

1-amino(mercaptopropyl)ethylphosphonic acid,

2-amino-1-(mercaptopropyl)ethylphosphonic acid, and

1-amino-1-(mercaptooctyl)butylphosphonic acid.

The compounds with sulfonic and/or phosphonic acid function forming the anionic units (a1) of the polyurethanes of the present invention may also be polymers.

These anionic polymers with sulfonic and/or phosphonic acid function may be obtained, in one step, by free-radical, anionic or cationic copolymerization, and in particular by ring opening, or by polycondensation of nonionic monomers and of anionic monomers carrying sulfonic acid or phosphonic acid units.

There may be mentioned by way of example anionic monomers which can be used for the free-radical polymerization of sodium styrenesulfonate.

The polymers may also be obtained in two steps, that is to say by polymerization or polycondensation of nonionic monomers, followed by the grafting of units with sulfonic or phosphonic acid function.

The nonionic monomers which can be used are for example vinyl monomers, ethylene oxide or propylene oxide. The polymers obtained by polycondensation may be polyesters, polyamides or polyurethanes.

The sulfonic acid or phosphonic acid functional groups may also be introduced by initiators carrying these functions.

The groups with labile hydrogen may be introduced by monomers, initiators or chain terminating agents carrying such groups.

There may be mentioned for example a diol used as initiator in the polymerization of propylene oxide by ring opening.

The weight-average molecular mass of these polymers with sulfonic and/or phosphonic acid functions is preferably between 200 and 10 000, and more preferably between 400 and 5000.

There may be mentioned by way of example of such appropriate polymers with sulfonic and/or phosphonic acid function the polymers of formula

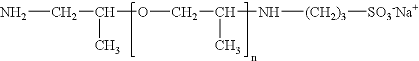

in which n is between 2 and 100. A polymer of this type in which n=7 is marketed under the name “Polypropylene glycol diamine sulfopropylated Na salt” by the company Rachig.

The second type of units forming the polyurethanes of the present invention are nonionic monomer or polymer units, called units (a2), carrying at their ends reactive functions with labile hydrogen.

While the presence of the anionic units (a1) and of the units (b) derived from diisocyanates is obligatory in the polyurethanes of the present invention, that of the nonionic units (a2) is optional. These nonionic units may be derived from monomers or from polymers.

There may be mentioned by way of examples of monomer compounds capable of forming the nonionic units (a2) neopentyl glycol, 1,6-hexanediol, 1,4-butanediol or aminoethanol.

The nonionic polymers capable of forming the units (a2) are chosen for example from polyethers, polyesters, polysiloxanes, copolymers of ethylene and butylene, polycarbonates, polyalkyl (meth)acrylates and fluorinated polymers.

The polyethers are most particularly preferred, and among them poly(tetramethylene oxide).

The weight-average molar mass of these nonionic polymers is preferably between 400 and 10 000 and more particularly between 400 and 5000. The elastic property of the polyurethanes of the present invention is linked to the simultaneous presence, in the polymer, of a certain fraction of polymer sequences having a glass transition temperature less than 10° C., and a certain fraction of units forming sequences which have a glass transition temperature greater than room temperature.

The sequences having a glass transition temperature less than 10° C., also called “soft” sequences, are formed by the anionic polymers and/or the nonionic polymers described above.

The viscoelastic properties of the anionic polyurethanes of the present invention are particularly advantageous when the units (a1) or (a2) are derived from polymers having a glass transition temperature less than 0° C. and better still less than −10° C.

The sequences with Tg greater than 20° C., also called “rigid” sequences, exist, at room temperature, in the glassy state and thus form physical crosslinking nodes of the three-dimensional polymer network.

The applicant has observed that the elasticity of the polyurethanes of the present invention is satisfactory when the fraction of anionic or nonionic polymer units having a glass transition temperature less than 10° C. represents from 20 to 90%, preferably from 20 to 80%, and in particular from 20 to 70% of the total weight of the polyurethanes of the present invention.

The diisocyanates forming the units (b) include aliphatic, alicyclic or aromatic diisocyanates.

Preferred diisocyanates are chosen from methylene-diphenyl diisocyanate, methylenecyclohexane diisocyanate, isophorone diisocyanate, toluene diisocyanate, naphthalene diisocyanate, butane diisocyanate and hexyl diisocyanate. These diisocyanates may of course be used alone or in the form of a mixture of two or more diisocyanates.

The elastic property of the anionic polyurethanes of the present invention is due to the fact that these polymers have at least two different glass transition temperatures (Tg), at least one of these Tg being less than 10° C. and at least another being greater than or equal to 20° C.

The physical parameter characterizing the viscoelastic properties of the above anionic polyurethanes is their tensile recovery. This recovery is determined by a tensile creep test consisting in rapidly stretching a test piece to a predetermined degree of elongation, and then in releasing the stress and measuring the length of the test piece.

The creep test used to characterize the anionic polyurethanes with elastic property of the present invention is carried out in the following manner:

There is used, as test piece, a polyurethane film having a thickness of 500±50 μm, cut into 80 mm×15 mm strips. This copolymer film is obtained by drying, at a temperature of 22±2° C. and at a relative humidity of 50±5%, a solution or dispersion at 3% by weight of said polyurethane in water and/or ethanol.

Each strip is fixed between two jaws 50±1 mm apart, and is stretched at a speed of 20 mm/minute (under the temperature and relative humidity conditions above) up to an elongation of 50% (εₘₐₓ), that is to say up to 1.5 times its initial length. The stress is then released by imposing a speed of return equal to the tensile speed, that is 20 mm/minute, and the elongation of the test piece (expressed as a percentage relative to the initial length) immediately after returning to zero loading (εᵢ) is measured.

The instantaneous recovery (Rᵢ) is calculated using the following formula:

Rᵢ(%)=((εₘₐₓ−εᵢ)/εₘₐₓ)×100

The anionic polyurethanes with elastic property of the present invention preferably have an instantaneous recovery (Rᵢ), measured under the conditions indicated above, between 5% and 100%, in particular between 20% and 100% and ideally between 35 and 100%.

The glass transition temperatures (Tg) of the polymers forming the units (a1) or (a2) and of the anionic polyurethanes of the present invention are measured by differential scanning calorimetry (DSC) according to the ASTM D3418-97 standard.

The instantaneous recovery, and consequently the viscoelastic properties of the polyurethanes of the present invention, depends on the proportions of the different units (a1), (a2) and (b) in the polymer.

The fraction of units (a1) should be sufficient to confer on the polymers their negative charge responsible for their good capacity to dissolve or to be dispersed in polar solvents such as water and alcohols.

The anionic polyurethanes of the present invention preferably have an anionic charge level between 0.1 and 15 milliequivalents per gram (meq/g), more preferably between 0.1 and 10 meq/g, and most particularly between 0.1 and 5 meq/g.

In terms of fraction by weight, the units (a1) represent in particular from 1 to 90% and preferably from 5 to 60% by weight, and the units (a2) advantageously represent from 0 to 90% and preferably from 40 to 70% by weight of the total polymer.

The units (b) are present in an essentially stoichiometric quantity relative to the sum of the units (a1) and (a2). Indeed, the production of polyurethanes having high molar masses assumes a number of isocyanate functions which is practically identical to the number of functions with labile hydrogen. Persons skilled in the art will know how to choose a possible molar excess of either type of function in order to adjust the molar mass to the desired value.

As indicated above, the anionic polyurethanes with elastic property may be incorporated into numerous cosmetic compositions of which they improve the cosmetic properties.

The quantity of polyurethane present in the various compositions of course depends on the type of composition and the desired properties and may vary within a very broad range, generally between 0.5 and 90% by weight, preferably between 1 and 50% by weight, relative to the final cosmetic composition.

When the anionic polyurethanes with elastic property are incorporated into hair lacquers, their concentration is generally between 0.5 and 15% by weight. In nail varnishes, they generally represent from 0.5 to 40% by weight of the composition, and makeup compositions for the skin, the lips and superficial both growths generally contain 0.5 to 20% by weight of the polyurethanes of the present invention.

It is also possible to envisage the use of the anionic polyurethanes with elastic property of the present invention in pure form, for example in order to form a protective film on the nails.

### EXAMPLE 1

Synthesis of an Anionic Polyurethane With Elastic Property

The following monomers and solvent are introduced into a thermostated reactor equipped with a mechanical stirring system and a condenser:

1 mol of poly(tetramethylene oxide) having a weight-average molar mass equal to 1400,

2 mol of 1,4-butanediol,

a quantity of THF such that the concentration of diol type monomers is equal to 75% by weight.

The mixture is heated, with stirring, to a temperature of 70° C., and then 5.15 mol of isophorone diisocyanate are introduced dropwise, with stirring, over a period of about 1 hour. During this addition, an increase in temperature up to the reflux temperature of the solvent is observed. This reflux is maintained for 4 hours, and then there are added over a period of 30 minutes, 2 mol of a diamine with a sulfo group of formula:

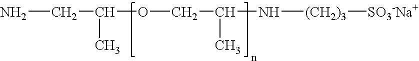

where n=7.

A sample, whose IR absorption spectrum is plotted in order to monitor the disappearance of the band corresponding to the isocyanate functions (2260 cm⁻¹) is collected at regular intervals.

When the absorption band for the —NCO functions no longer decreases, which is generally the case after about 5 hours, the reaction mixture is allowed to cool to room temperature, and then diluted with acetone to a polymer concentration of about 40% by weight.

20 ml of ethanol are then added to the mixture obtained in order to deactivate the residual —NCO functions and the stirring is continued at room temperature until complete disappearance of the —NCO functions, that is to say of the IR absorption band at 2260 cm⁻¹, is obtained.

The organic phase is then removed by distillation under vacuum at a temperature of 40° C.

After removing the organic phase, a sufficient quantity of water is added to the aqueous solution of the polymer in order to obtain a polymer concentration in the water of about 25% by weight.

### EXAMPLE 2

Measurement of the Instantaneous Recovery

Polyurethane films are prepared from dispersions at. 3% by weight of the polyurethane of Example 1 in a water/ethanol (1/2) mixture. The instantaneous recovery (expressed as %) is measured under the following conditions:

thickness of the film: 500+50 μm,

dimension of the strips of 80 mm×15 mm

drying conditions: 22±2° C., relative humidity of 50±5%,

distance between two jaws: 50±1 mm,

stretching speed=return speed: 20 mm/minute.

The instantaneous recovery (Rᵢ) is calculated using the following formula:

Rᵢ(%)=((εₘₐₓ−εᵢ)/εₘₐₓ)×100

The instantaneous recovery of the polyurethane of Example 1, measured under these conditions, is 70%.

1-23. (canceled)

24. An anionic polyurethane having an elastic property comprising:
an anionic unit derived from at least one monomeric or polymeric compound, wherein said monomeric or polymeric compound comprises at least one of sulfonic acid and phosphonic acid and at least two reactive functions containing at least one labile hydrogen; and
a unit derived from at least one diisocyanate,
said anionic polyurethane optionally comprising a nonionic unit derived from at least one nonionic monomeric or nonionic polymeric compound, wherein said nonionic monomeric or nonionic polymeric compound comprises at least two reactive functions containing at least one labile hydrogen; and
wherein at least one of said anionic unit and said optional nonionic unit is derived from at least one polymer having a glass transition temperature measured by differential scanning calorimetry less than 10° C., said polymer comprising from about 20% to about 90% of the total weight of the anionic polyurethane.

25. The anionic polyurethane according to claim 24, wherein at least one of said anionic unit and said optional non-ionic unit is derived from at least one polymer having a glass transition temperature less than 0° C.

26. The anionic polyurethane according to claim 25, wherein at least one of said anionic unit and said optional non-ionic unit is derived from at least one polymer having a glass transition temperature less than −10° C.

27. The anionic polyurethane according to claim 24, wherein said at least one polymer having a glass transition temperature less than 10° C. comprises from about 20% to about 80% of the total weight of the anionic polyurethane.

28. The anionic polyurethane according to claim 27, wherein said at least one polymer having a glass transition temperature less than 10° C. comprises from about 20% to about 70% of the total weight of the anionic polyurethane.

29. The anionic polyurethane according to claim 24, wherein said elastic property comprises an instantaneous recovery ranging from about 5% to about 100%.

30. The anionic polyurethane according to claim 29, wherein said instantaneous recovery ranges from about 35% to about 100%.

31. The anionic polyurethane according to claim 24, wherein said anionic unit is derived from compounds chosen from at least one of the following formulas: 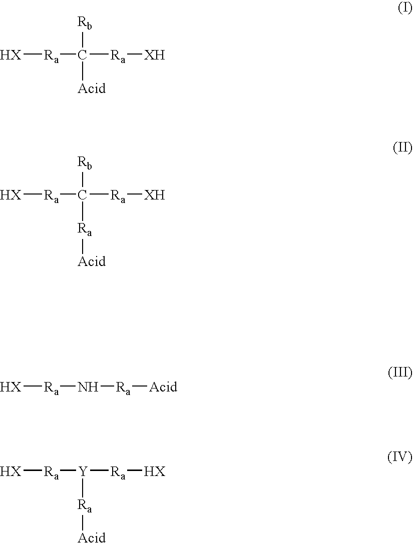
wherein said Acid is chosen from sulfonic acid groups and phosphonic acid groups, optionally protonated or in salt form,
Rₐ, which can be identical or different, is chosen from direct bonds, linear C₁₋₆ alkylene groups, branched C₁₋₆ alkylene groups, C₃₋₆ cycloalkylene groups, and arylene groups, wherein Rₐ is unsubstituted or substituted with at least one halogen atom or at least one heteroatom chosen from O, P, and S,
R_{b} is chosen from hydrogen and alkyl groups, wherein Rb is unsubstituted or substituted with at least one heteroatom chosen from O, P and S,
Y is chosen from saturated cyclic C₅₋₁₀ groups, unsaturated cyclic C₅₋₁₀ groups, and aromatic cyclic C₅₋₁₀ groups, wherein Y is unsubstituted or substituted with at least one heteroatom chosen from O, P and S, and
X, which can be identical or different, is chosen from oxygen atoms, sulfur atoms, NH, and NR_{c} groups, wherein R_{c} is a C₁₋₆ alkyl group.

32. The anionic polyurethane according to claim 31, wherein X is chosen from NH and NR_{c} groups, where R_{c} is a C₁₋₆ alkyl group.

33. The anionic polyurethane according to claim 31, wherein the compounds of formula (I) to (IV) are chosen from:
1,1-diaminomethanesulfonic acid,
diaminobenzenesulfonic acid,
1,1-di(hydroxymethyl)ethanesulfonic acid,
1,1-di(hydroxyethyl)ethanesulfonic acid,
1,1-di(hydroxymethyl)propanesulfonic acid,
1,1-di(hydroxymethyl)methanesulfonic acid,
1,1-di(hydroxypropyl)ethanesulfonic acid,
1,1-di(hydroxyoctyl)ethanesulfonic acid,
3-(2,3-dihydroxypropoxy)propanesulfonic acid,
3-[2,2-bis(hydroxymethyl)butoxy]-2-methyl-1-propanesulfonic acid,
1,1-di(aminoethyl)ethanesulfonic acid,
1,1-diaminomethanesulfonic acid,
1,1-diaminopropanesulfonic acid,
1,2-diaminopropanesulfonic acid,
1,1-di(mercaptoethyl)ethanesulfonic acid,
1,1-dimercaptomethanesulfonic acid,
1,1-dimercaptopropanesulfonic acid,
1,2-dimercaptohexanesulfonic acid,
1-amino-1-hydroxymethylethanesulfonic acid,
1-amino-1-hydroxyethylethanesulfonic acid,
1-amino-1-hydroxymethylpropanesulfonic acid,
1-amino-1-hydroxymethylmethanesulfonic acid,
1-amino-1-hydroxypropylethanesulfonic acid,
2-amino-1-hydroxypropylethanesulfonic acid,
1-amino-1-hydroxyoctylethanesulfonic acid,
1-hydroxymethyl-1-mercaptoethanesulfonic acid,
11-hydroxyethyl-2-mercaptoethylethanesulfonic acid,
1-hydroxymethyl-1-mercaptopropanesulfonic acid,
1-hydroxypropyl-1-mercaptomethanesulfonic acid,
1-hydroxypropyl-1-mercaptoethanesulfonic acid,
2-hydroxyethyl-1-mercaptomethylethanesulfonic acid,
1-hydroxybutyl-1-mercaptooctylethanesulfonic acid,
1-amino-1-mercaptomethylethanesulfonic acid,
1-amino-1-mercaptoethylethanesulfonic acid,
1-amino-1-mercaptomethylpropanesulfonic acid,
1-amino-1-mercaptomethylmethanesulfonic acid,
1-amino-1-mercaptopropylethanesulfonic acid,
2-amino-1-mercaptopropylethanesulfonic acid,
1-amino-1-mercaptooctylethanesulfonic acid,
1-amino-1-mercaptopropylethylphosphonic acid,
dimercaptomethylphosphonic acid,
dimercaptoethylphosphonic acid,
dimercaptopropylphosphonic acid,
1-amino-1-hydroxymethylethylphosphonic acid,
1-amino-1-(hydroxyethyl)ethylphosphonic acid,
1-amino-1-(hydroxymethyl)propylphosphonic acid,
1-amino-1-(hydroxymethyl)methylphosphonic acid,
1-amino-1-(hydroxypropyl)ethylphosphonic acid,
2-amino-1-(hydroxypropyl)ethylphosphonic acid,
1-amino-1-(hydroxyoctyl)ethylphosphonic acid,
1-hydroxymethyl-1-mercaptoethylphosphonic acid,
1-hydroxyethyl-2-(mercaptoethyl)ethylphosphonic acid,
1-hydroxymethyl-1-mercaptopropylphosphonic acid,
1-hydroxypropyl-1-mercaptomethylphosphonic acid,
1-hydroxypropyl-1-(mercaptobutyl)ethylphosphonic acid,
2-hydroxyethyl-1-(mercaptomethyl)ethylphosphonic acid,
1-hydroxybutyl-1-(mercaptooctyl)ethylphosphonic acid,
1-amino-1-(mercaptomethyl)ethylphosphonic acid,
1-amino-1-(mercaptoethyl)butylphosphonic acid,
1-amino-1-mercaptopropylphosphonic acid,
1-amino(mercaptopropyl)ethylphosphonic acid
2-amino-1-(mercaptopropyl)ethylphosphonic acid, and
1-amino-1-(mercaptooctyl)butylphosphonic acid.

34. The anionic polyurethane according to claim 24, wherein said anionic unit is derived from at least one polymer of the following formula 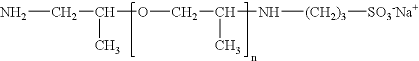
where n ranges from 2 to 100.

35. The anionic polyurethane according to claim 24, wherein said nonionic unit is derived from at least one monomer compound chosen from neopentyl glycol, 1,6-hexanediol, 1,4-butanediol, and aminoethanol.

36. The anionic polyurethane according to claim 24, wherein said nonionic unit is derived from at least one polymer chosen from polyethers, polyesters, polysiloxanes, copolymers of ethylene and butylene, polycarbonates, polyalkyl(meth)acrylates, and fluorinated polymers.

37. The anionic polyurethane according to claim 36, wherein said monomeric and polymeric compounds forming said nonionic unit have a weight-average molar mass ranging from about 200 to about 10,000.

38. The anionic polyurethane according to claim 37, wherein said nonionic monomeric and nonionic polymeric compounds forming said nonionic unit have a weight-average molar mass ranging from about 400 to about 5,000.

39. The anionic polyurethane according to claim 36, wherein said nonionic unit is derived from poly(tetramethylene oxide).

40. The anionic polyurethane according to claim 24, wherein said unit derived from at least one diisocyanate is chosen from methylenediphenyl diisocyanate, methylenecyclohexane diisocyanate, isophorone diisocyanate, toluene diisocyanate, naphthalene diisocyanate, butane diisocyanate, and hexyl diisocyanate.

41. The anionic polyurethane according to claim 24, wherein said anionic unit is present from about 1% to about 90% by weight of the total anionic polyurethane, said nonionic unit is present up to 90% by weight of the total anionic polyurethane, and said unit derived from at least one diisocyanate is present in an essentially stoichiometric quantity relative to the sum of the anionic units and nonionic units.

42. The anionic polyurethane according to claim 41, wherein said anionic unit is present from about 5% to about 60% by weight of the total anionic polyurethane.

43. The anionic polyurethane according to claim 41, wherein said nonionic unit is present from about 40% to about 70% by weight of the total anionic polyurethane.

44. The anionic polyurethane according to claim 24, having an anionic charge level ranging from about 0.1 to about 15 meq/g of the anionic polyurethane.

45. The anionic polyurethane according to claim 44, wherein said anionic charge level ranges from about 0.1 to about 10 meq/g of the anionic polyurethane.

46. The anionic polyurethane according to claim 45, wherein said anionic charge level ranges from about 0.1 to about 5 meq/g of the anionic polyurethane.

47. A cosmetic composition comprising at least one anionic polyurethane having an elastic property comprising:
an anionic unit derived from at least one monomeric or polymeric compound, wherein said monomeric or polymeric compound comprises at least one of sulfonic acid and phosphonic acid and at least two labile hydrogens; and
a unit derived from at least one diisocyanate,
said anionic polyurethane optionally comprising a nonionic unit derived from at least one nonionic monomeric or nonionic polymeric compound, wherein said nonionic monomeric or nonionic polymeric compound comprises at least two labile hydrogens; and
wherein at least one said anionic unit and said optional nonionic unit is derived from at least one polymer having a glass transition temperature less than 10° C., said polymer comprising from about 20% to about 90% of the total weight of the anionic polyurethane.

48. The cosmetic composition according to claim 47, wherein said cosmetic composition is a hair lacquer comprising from about 0.5% to about 15% by weight of the anionic polyurethane relative to the total weight of the cosmetic composition.

49. The cosmetic composition according to claim 47, wherein said cosmetic composition is a nail varnish comprising from about 0.5% to about 40% by weight of the anionic polyurethane relative to the total weight of the cosmetic composition.

50. The cosmetic composition according to claim 47, wherein said cosmetic composition is a makeup composition comprising from about 0.5% to about 20% by weight of the anionic polyurethane relative to the total weight of the cosmetic composition.

51. A method of using an anionic polyurethane having an elastic property comprising:
an anionic unit derived from at least one monomeric or polymeric compound, wherein said monomeric or polymeric compound comprises at least one of sulfonic acid and phosphonic acid and at least two reactive functions containing at least one labile hydrogen; and
a unit derived from at least one diisocyanate,
said anionic polyurethane optionally comprising a nonionic unit derived from at least one nonionic monomeric or nonionic polymeric compound, wherein said nonionic monomeric or nonionic polymeric compound comprises at least two reactive functions containing at least one labile hydrogen; and
wherein at least one of said anionic unit and said optional nonionic unit is derived from at least one polymer having a glass transition temperature measured by differential scanning calorimetry less than 10° C., said polymer comprising from about 20% to about 90% of the total weight of the anionic polyurethane, said method comprising incorporating said anionic polyurethane in a hair lacquer.

52. A method of using an anionic polyurethane having an elastic property comprising:
an anionic unit derived from at least one monomeric or polymeric compound, wherein said monomeric or polymeric compound comprises at least one of sulfonic acid and phosphonic acid and at least two reactive functions containing at least one labile
a unit derived from at least one diisocyanate,
said anionic polyurethane optionally comprising a nonionic unit derived from at least one nonionic monomeric or nonionic polymeric compound, wherein said nonionic monomeric or nonionic polymeric compound comprises at least two reactive functions containing at least one labile hydrogen; and
wherein at least one of said anionic unit and said optional nonionic unit is derived from at least one polymer having a glass transition temperature measured by differential scanning calorimetry less than 10° C., said polymer comprising from about 20% to about 90% of the total weight of the anionic polyurethane, said method comprising incorporating said anionic polyurethane in a nail varnish.

53. A method of using an anionic polyurethane having an elastic property comprising:
an anionic unit derived from at least one monomeric or polymeric compound, wherein said monomeric or polymeric compound comprises at least one of sulfonic acid and phosphonic acid and at least two reactive functions containing at least one labile hydrogen; and
a unit derived from at least one diisocyanate,
said anionic polyurethane optionally comprising a nonionic unit derived from at least one nonionic monomeric or nonionic polymeric compound, wherein said nonionic monomeric or nonionic polymeric compound comprises at least two reactive functions containing at least one labile hydrogen; and wherein at least one of said anionic unit and said optional nonionic unit is derived from at least one polymer having a glass transition temperature measured by differential scanning calorimetry less than 10° C., said polymer comprising from about 20% to about 90% of the total weight of the anionic polyurethane, said method comprising forming a protective film on nails.

54. A method of using an anionic polyurethane having an elastic property comprising:
an anionic unit derived from at least one monomeric or polymeric compound, wherein said monomeric or polymeric compound comprises at least one of sulfonic acid and phosphonic acid and at least two reactive functions containing at least one labile hydrogen; and
a unit derived from at least one diisocyanate,
said anionic polyurethane optionally comprising a nonionic unit derived from at least one nonionic monomeric or nonionic polymeric compound, wherein said nonionic monomeric or nonionic polymeric compound comprises at least two reactive functions containing at least one labile hydrogen; and
wherein at least one of said anionic unit and said optional nonionic unit is derived from at least one polymer having a glass transition temperature measured by differential scanning calorimetry less than 10° C., said polymer comprising from about 20% to about 90% of the total weight of the anionic polyurethane, said method comprising incorporating said anionic polyurethane in a makeup composition.

55. The method according to claim 54, wherein said makeup composition is applied to at least one of the skin, lips, and superficial body growths.
